(19) United States
(12) Patent Application Publication
(10) Pub. No.: US 2023/0062487 A1
(43) Pub. Date: Mar. 2, 2023

(54) SUSTAINED-RELEASE MATRICES FOR ADVENTITIAL OR PERIADVENTITIAL NEURAL ABLATION AND USES THEREOF

(71) Applicant: W. L. Gore & Associates, Inc., Newark, DE (US)

(72) Inventors: Charles D. Claude, Flagstaff, AZ (US); Robert L. Cleek, Flagstaff, AZ (US); Paul D. Drumheller, Flagstaff, AZ (US); Andre T. Stevenson, Flagstaff, AZ (US)

(21) Appl. No.: 17/795,099

(22) PCT Filed: Jan. 22, 2021
(86) PCT No.: PCT/US2021/014717

§ 371 Date: Jul. 25, 2022

Related U.S. Application Data
(60) Provisional application No. 62/965,551, filed on Jan. 24, 2020.

(51) Int. Cl. A61K 9/51; A61K 31/337

(52) U.S. Cl. CPC A61K 9/5146; A61K 31/337; A61K 9/5138; A61K 9/5123

(57) ABSTRACT

A denervation formulation including a denervation drug incorporated into a sustained-release matrix. The sustained-release matrix may include a polycarbonate and a fluoropolymer. The sustained-release matrix may form a plurality of particles to encapsulate the denervation drug. The denervation formulation may be delivered to a patients autonomic neural tissue, including but not limited to a renal sympathetic nerve, a carotid nerve, a pulmonary nerve, and/or a cardiac sympathetic nerve. Upon release, the denervation drug may ablate the patients autonomic neural tissue for treatment of cardiac disease, including but not limited to hypertension, heart failure, and/or atrial and ventricular tachycardia.

### CROSS-REFERENCE TO RELATED APPLICATION

This application is a national phase application of PCT Application No. PCT/US2021/014717, internationally filed on Jan. 22, 2021, which claims the benefit of Provisional Application No. 62/965,551, filed Jan. 24, 2020, which are incorporated herein by reference in their entireties for all purposes.

### FIELD

The present disclosure relates generally to autonomic neural ablation, and more specifically to a sustained-release formulation and method for autonomic neural ablation for the treatment of cardiovascular disease.

### BACKGROUND

The autonomic nervous system (ANS), which includes the sympathetic nervous system, is interconnected with the cardiovascular system. Certain cardiovascular disease states originate from the neurohormonal response to renal sympathetic nerve activation, including hypertension, heart failure, type II diabetes and atrial and/or ventricular tachycardia. Other sympathetic neural systems such as those associated with the hepatic and pulmonary system may be targeted for fatty liver disease or pulmonary arterial hypertension. The sympathetic nervous system is also interconnected with the digestive system and may impact digestive functions including but not limited to resting metabolic rate and dissipation of consumed calories, so sympathetic nerve activation may lead to weight gain. One sympathetic nerve treatment includes oral medications, but some patients are unresponsive and about half of patients fail to take such oral medications at all or properly. Another sympathetic nerve treatment includes energy-based ablation procedures, but anatomical features may limit the depth and uniformity of such procedures. Yet another sympathetic nerve treatment includes acute drug delivery, but the amount of drug delivered may be limited by nonspecific tissue toxicity.

### SUMMARY

A denervation formulation is disclosed including a denervation drug incorporated into a sustained-release matrix. The sustained-release matrix may include a polycarbonate or a fluoropolymer. The sustained-release matrix may form a plurality of microparticles and/or nanoparticles to encapsulate the denervation drug. The denervation formulation may be delivered to a patient's autonomic neural tissue, including but not limited to a renal sympathetic nerve, a carotid nerve, a pulmonary nerve, a hepatic nerve and/or a cardiac sympathetic nerve. Upon release, the denervation drug may ablate the patient's autonomic neural tissue for treatment of cardiac disease, including but not limited to hypertension, heart failure, and/or atrial and ventricular tachycardia, treatment of bariatric conditions, or treatment of other disease states.

According to one example (“Example 1”), a formulation is provided including a sustained-release matrix comprising at least one of a polycarbonate and a fluoropolymer; and a denervation drug incorporated into the sustained-release matrix.

According to another example (“Example 2”), a denervation method is provided including delivering a denervation formulation to an autonomic neural tissue of a patient with a cardiovascular disease, the denervation formulation comprising a denervation drug incorporated into a sustained-release matrix, gradually releasing the denervation drug into the autonomic neural tissue of the patient, and ablating the patient's autonomic neural tissue.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a schematic view of a denervation formulation in a delivery device, the denervation formulation including a denervation drug incorporated into a sustained-release matrix in accordance with an embodiment;

FIG. 2 is a flow chart of a denervation method in accordance with an embodiment;

FIG. 3 are scanning electron microscope (SEM) images of denervation microparticles in accordance with Example A;

FIG. 4 is a graphical representation of the release profile of a denervation drug from denervation microparticles in accordance with Example A;

FIG. 5 is a graphical representation of the volume distribution of TFE-VOH nanoparticles in accordance with Example E;

FIG. 6 is a FTIR spectra for a TFE-VOH stock solution (A) and nanoparticles (B) in accordance with Example E.

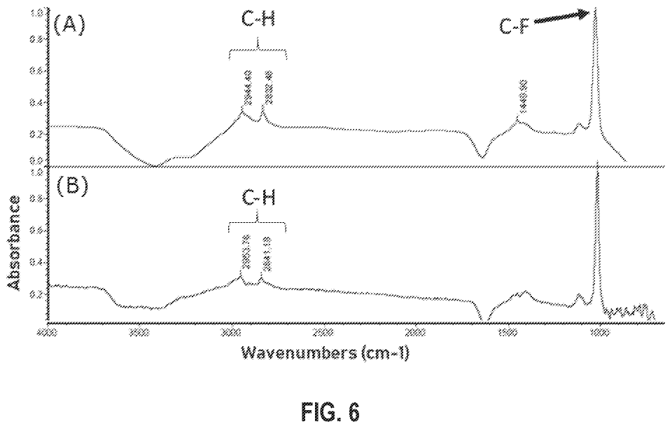

This disclosure is not meant to be read in a restrictive manner. For example, the terminology used in the application should be read broadly in the context of the meaning those in the field would attribute such terminology.

With respect terminology of inexactitude, even when the terms “about” and “approximately” are not used any stated value referring to a measurement includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error or minor adjustments made to optimize performance, for example.

The foregoing Examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### DETAILED DESCRIPTION

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale but may be exaggerated to illustrate various aspects of the present disclosure, and in that regard, the drawing figures should not be construed as limiting.

### Denervation Formulation

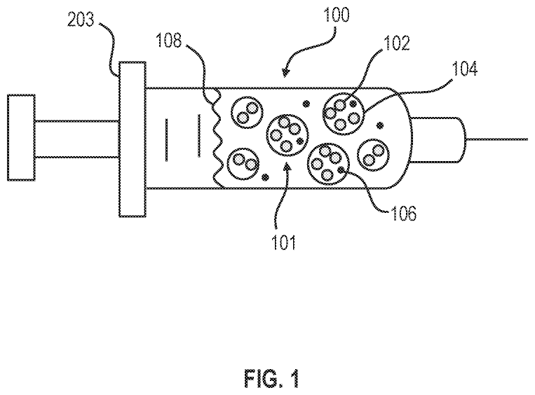

With reference to FIG. 1, a denervation formulation 100 is disclosed for delivery to a patient's autonomic neural tissue. The autonomic neural tissue may be located in the adventitial or periadventitial region of a vascular structure, a cardiovascular structure, or another organ. The autonomic neural tissue may include, for example, a renal sympathetic nerve, a carotid nerve, a pulmonary nerve, and/or a cardiac sympathetic nerve.

The denervation formulation 100 includes a plurality of particles 101, which may be microparticles (e.g., microspheres) and/or nanoparticles, with each particle 101 including a denervation drug 102 incorporated into a sustained-release matrix 104 (which may also be referred to as a controlled-release matrix). The denervation formulation 100 may also include one or more optional excipients 106 and a delivery fluid 108. Each ingredient of the denervation formulation 100 is described further below.

The denervation drug 102 is a neurotoxin configured to ablate (i.e., inhibit or destruct) the patient's autonomic neural tissue and interrupt or otherwise hinder the transmission of neural signals from the autonomic neural tissue. Suitable denervation drugs 102 include but are not limited to paclitaxel (PTX), suramin, digoxin, altretamine, oxaliplatin, vincristine, vinblastine, cisplatin, carboplatin, bortezomib, and etoposide, as well as analogs and salts thereof.

The sustained-release matrix 104 may encapsulate the denervation drug 102 to form the particle 101, as shown in FIG. 1. In certain embodiments, the particles 101 may be microparticles having an average diameter (e.g., a volume-based mean diameter My in accordance with Example E below) from 1 μm to 20 μm, such as 1 μm, 2 μm, 4 μm, 6 μm, 8 μm, 10 μm, 12 μm, 14 μm, 16 μm, 18 μm, or 20 μm. In other embodiments, the particles may be nanoparticles having an average diameter between 40 nm and 1000 nm (1 μm), such as 50 nm, 100 nm, 200 nm, 300 nm, 400 nm, 500 nm, 600 nm, 700 nm, 800 nm, or 900 nm. The sustained-release matrix 104 may also be provided in other shapes and sizes.

The particles 101 of the instant invention are durable. As is known to the art, durable microparticles and nanoparticles do not immediately dissolve into their molecular entities after administration or immediately degrade through normal biodegradation mechanisms within the body (J L Weaver et al., Evaluating the potential of gold, silver, and silica nanoparticles to saturate mononuclear phagocytic system tissues under repeat dosing conditions, Particle and Fibre Toxicology, Vol 14, No. 1, Article 25). Rather, durable microparticles and nanoparticles remain in the particulate state during administration, distribution, accumulation, or elimination. In certain embodiments, the microparticles and nanoparticles when implanted remain durable for a time period of 7 days to 180 days, such as 7 days, 20 days, 40 days, 60 days, 80 days, 100 days, 120 days, 140 days, 160 days, or 180 days.

Examples of suitable sustained-release matrices 104 include polycarbonates and fluoropolymers, as described further below. Furthermore, the sustained-release matrix may be a solid, a gel, or combinations thereof.

The sustained-release matrix 104 is a material configured to release the denervation drug 102 gradually over an extended time period of several hours, several days, weeks, or months following delivery to the patient. The extended time period may be 1 day to 180 days, such as 1 day, 10 days, 20 days, 40 days, 60 days, 80 days, 100 days, 120 days, 140 days, 160 days, or 180 days. In certain embodiments, the extended time period is 5 days, 7 days, 9 days, 11 days, 13 days, or 15 days, for example.

The sustained-release matrix 104 may be designed to control the gradual release rate of the denervation drug 102 between a minimum rate sufficient to achieve neural ablation and a maximum rate that ensures patient safety by avoiding nonspecific tissue toxicity. For example, the gradual release rate may be 0.1 μg/day to 6 mg/day, such as 0.1 μg/day, 0.5 μg/day, 1 μg/day, 1.5 μg/day, or 2 μg/day. In some embodiments, the gradual release rate may be 1 μg/day to 800 μg/day, 1 μg/day to 700 μg/day, 1 μg/day to 600 μg/day, 1 μg/day to 500 μg/day, 1 μg/day to 400 μg/day, 1 μg/day to 300 μg/day, 100 μg/day to 700 μg/day, 200 μg/day to 700 μg/day, 300 μg/day to 700 μg/day, 400 μg/day to 700 μg/day, 500 μg/day to 700 μg/day, 100 μg/day to 500 μg/day, 100 μg/day to 400 μg/day, or 100 μg/day to 300 μg/day. For example, the gradual release rate may be 300 μg/day, 350 μg/day, 400 μg/day, 450 μg/day, 500 μg/day, 550 μg/day, 600 μg/day, 650 μg/day, 700 μg/day, or 750 μg/day. The gradual release rate may vary based on the selected denervation drug 102, the exact or approximate anatomical site, the patient's weight, the patient's age, the patient's overall health, and other factors. The gradual release rate may be steady over time or may vary over time (e.g., a faster initial rate (i.e., burst) followed by a slower final rate).

Because the denervation drug 102 is released gradually, each dose of the denervation formulation 100 that is delivered to the patient may contain a large amount of the denervation drug 102. For example, each dose of the denervation formulation 100 depending on the drug may contain 30 to 400 μg of the denervation drug 102 in one case and 7 mg to 30 mg in another, such as 50, 100 μg, 400 ug in the case of one drug and 7 mg, 10 mg and 30 mg in another case of the denervation drug 102. In some embodiments, the denervation drug 102 may account for 1 wt. %, 5 wt. %, 10 wt. %, 15 wt. %, 20 wt. %, or more of each particle 101.

The optional excipient(s) 106 may be configured to alter the release rate of the denervation drug 102 from the denervation formulation 100, to increase tissue permeability of the denervation drug 102, and/or to interact with surface receptors that are neural cell specific to increase the potency of the denervation drug 102. Suitable excipients 106 include cyclodextrin, polyethylene glycol (PEG), poloxamers, polyvinyl alcohol (PVA), dodecylsulfoxide, decylmethylsulfoxide, calcium salicylate or any other organo-calcium sources, and sodium glutamate, for example. The excipient(s) 106 may be present within the particles 101, around the particles 101, and/or in the delivery fluid 108. In some embodiments, the excipients 106 may account for 1 wt. %, 5 wt. %, 10 wt. %, 15 wt. %, 20 wt. %, 25 wt. %, or more of the denervation formulation 100.

The delivery fluid 108 may be mixed with the particles 101 to produce an injectable denervation formulation 100. The particles 101 may be suspended, dissolved, or otherwise mixed with the delivery fluid 108. The delivery fluid 108 may maintain a non-inflammatory surrounding state. The delivery fluid 108 may include, for example, water, phosphate buffered saline (PBS), parenteral oils, triacetin (1,2,3-triacetoxypropane), acetyltributyl citrate, triethyl citrate, tributyl citrate, acetyl triethyl citrate, 1-butanol, 2-butanol, butyl acetate, dimethylsulfoxide (DMSO), tert-butylmethyl ether, formic acid, 3-methyl-1-butanol, propylene glycol, polyethylene oxide, and combinations thereof.

### Polycarbonate Sustained Release Matrix

An example of a suitable polycarbonate sustained-release matrix 104 for the denervation formulation 100 is a bioabsorbable trimethylene carbonate (TMC) based polymer, which may include a TMC moiety polymerized with a polylactic acid (PLA) moiety and/or a polyglycolic acid (PGA) moiety.

In one embodiment, the TMC-based polymer may be a poly(lactic acid-TMC) copolymer, hereinafter “PLA:TMC”. The PLA:TMC copolymer may be synthesized using methods well known to the art, such as by combining TMC monomers with suitable comonomers of lactic acid, such as L-Lactic acid comonomers creating poly(L,Lactic acid-TMC) hereinafter “L-PLA:TMC”; D-Lactic acid comonomers creating poly(D,Lactic acid-TMC) hereinafter “D-PLA:TMC”; and comonomers of L-lactic acid and D-lactic acid and TMC creating Poly(DL,Lactic acid-TMC) hereinafter “D,L-PLA:TMC”. The PLA:TMC copolymers may have a weight ratio of D-PLA to TMC of 55% to 45% (55:45) or 75% to 25% (75:25), L-PLA to TMC of 55% to 45% (55:45) or 75% to 25% (75:25), and D,L-PLA to TMC of 50% to 50% (50:50) or 75% to 25% (75:25) (all based on weight). In some aspects, the PLA:TMC copolymer may comprise from 45 to 60 wt. % PLA and from 40 to 55 wt. % TMC. The PLA:TMC copolymer may have a number average molecular weight greater than 20,000 g/mol and a solubility in the delivery fluid 108 greater than 2 wt. %.

In another embodiment, the TMC-based polymer may be a poly(lactic and glycolic acid-TMC) terpolymer, hereinafter “PLA:PGA:TMC”. The PLA:PGA:TMC terpolymer may be synthesized using methods well known to the art, such as by combining TMC monomers, comonomers of lactic acid (as discussed above), and comonomers of glycolic acid. The PLA:PGA:TMC terpolymer may comprise from 3-19 wt. % PGA and may comprise PLA:TMC in a weight ratio from 3.25:1 to 0.75:1. The PLA:PGA:TMC terpolymer may have a weight ratio of D-PLA to TMC of 3.25:1 to 0.75:1, L-PLA to TMC of 3.25:1 to 0.75:1, or D,L-PLA to TMC of 3.25:1 to 0.75:1. The PLA:PGA:TMC terpolymer may have a number average molecular weight of 25,000 to 40,000 g/mol.

In another embodiment, the sustained-release matrix 104 may comprise an amphiphilic block copolymer, and/or may comprise additional additives, surfactants, or compounds to provide amphiphilic characteristics to the sustained-release matrix 104. Examples of amphiphilic block copolymers may comprise hydrophobic and hydrophilic domains or blocks. The hydrophobic domain/block may comprise lactide, glycolide, trimethylene carbonate and combinations thereof. The hydrophilic domain/block may consist of polyethylene glycol or hydrophilic naturally derived polymers such as saccharides including heparin, or block polymers thereof with polyethyleneglycol.

The PLA:PGA:TMC terpolymer may be synthesized using methods well known to the art, such as by combining TMC monomers, comonomers of lactic acid (as discussed above), and comonomers of glycolic acid, while using a terminal hydroxyl of the hydrophilic polymer as the initiator for the ring opening polymerization The PLA:PGA:TMC terpolymer may comprise from 3-19 wt. % PGA and may comprise PLA:TMC in a weight ratio from 3.25:1 to 0.75:1. The PLA:PGA:TMC terpolymer may have a weight ratio of D-PLA to TMC of 3.25:1 to 0.75:1, L-PLA to TMC of 3.25:1 to 0.75:1, or D,L-PLA to TMC of 3.25:1 to 0.75:1. The PLA:PGA:TMC terpolymer may have a number average molecular weight of 25,000 to 40,000 g/mol. The hydrophilic domain may have a molecular weight between 600 g/mol to 20,000 g/mol. If a hydrophobic-hydrophilic block copolymer with polyethylene glycol-saccharide blocks are used the hydrophobic-polyethylene glycol can be used as a substrate for saccharide coupling as known in the art.

In one example, the denervation formulation 100 may be formed by: dissolving the denervation drug 102 and the TMC-based polymer optionally comprising an absorbable amphiphilic polymer in an organic solvent (e.g., dichloromethane (DCM)/methanol); emulsifying the organic solution in an aqueous solution (e.g., PVA/water) to form particles 101 containing the denervation drug 102 and the TMC-based polymer as the sustained-release matrix 104; isolating the particles 101; and drying the particles 101. The particles 101 may then be mixed into the delivery fluid 108 for injection into the patient. The denervation formulation 100 may be stored and/or produced directly within vials, syringes, or any other suitable container.

A denervation formulation 100 with a TMC-based sustained-release matrix 104 is further exemplified in Example A below.

### Fluoropolymer Sustained Release Matrix

Another example of a suitable sustained-release matrix 104 for the denervation formulation 100 is a fluoropolymer including a tetrafluoroethylene (TFE) moiety and a vinyl moiety, wherein the vinyl moiety comprises at least one functional group selected from acetate, alcohol, amine, and amide. Suitable fluoropolymers include poly(tetrafluoroethylene-co-vinyl acetate) (TFE-VAc), poly(tetrafluoroethylene-co-vinyl alcohol) (TFE-VOH), and/or poly(tetrafluoroethylene-co-vinyl alcohol-co-vinyl[aminobutyraldehyde acetal]) (TFE-VOH-AcAm), for example. The fluoropolymer may have a TFE moiety mole content of at least 15%, such as 15.5% to 23.5%, and a vinyl moiety mole content of at least 76%, such as 76.5% to 84.5%. However, other TFE moiety and vinyl moiety mole contents are also contemplated.

In this example, the denervation formulation 100 may be formed by: dissolving the denervation drug 102 in water; dissolving the fluoropolymer in an organic solvent; emulsifying the water solution with the organic solution (where such emulsification is described in U.S. Pat. No. 9,731,017); and then hardening the emulsion to form a solid or gel containing the denervation drug 102 and the TFE-based fluoropolymer as the sustained-release matrix 104. In certain embodiments, the TFE-based sustained-release matrix 104 may be formed into particles 101 as described above. The particles 101 may then be mixed into the delivery fluid 108 for injection into the patient.

Suitable fluoropolymer compositions such as TFE-VOH may spontaneously form dispersed nanoparticles upon addition into an aqueous solvent. The hydrophobicity of TFE coupled with vinyl moiety hydrophilicity may enable simple nanoparticle formation due to thermodynamics. Specifically, it may be entropically favorable for TFE-VOH to precipitate into nanoparticles, which would lower the interfacial energy between the hydrophobic TFE and the aqueous environment leading to VOH likely residing on the nanoparticle perimeter. TFE-VOH that did not form nanoparticles may be of high molecular weight, which may be separated from solution such as by centrifugation or filtration. Additionally, the vinyl moiety may act as an electrostatic barrier enabling charge repulsion of the nanoparticles ultimately preventing agglomeration.

A TFE-VOH nanoparticle formulation is further exemplified in Example E below.

### Denervation Method

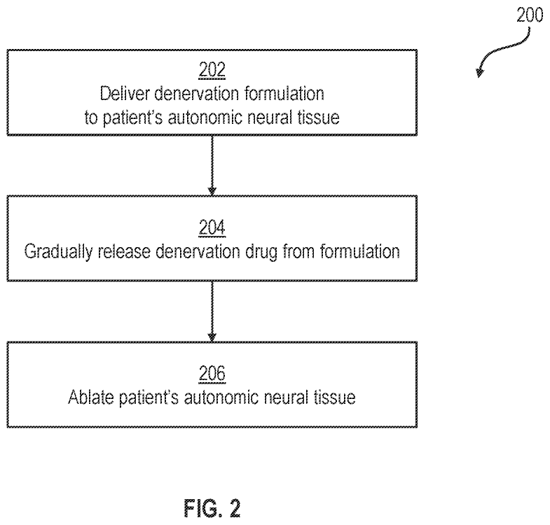

With reference to FIG. 2, a denervation method 200 may be used to treat a patient suffering from cardiovascular disease including but not limited to hypertension (e.g., systolic-diastolic hypertension, isolated diastolic hypertension, pulmonary arterial hypertension), heart failure, and/or atrial and ventricular tachycardia, for example.

In step 202, the denervation formulation 100 may be injected, delivered to the periadventitial region using a catheter or otherwise delivered in vivo to the patient's autonomic neural tissue. The autonomic neural tissue may be located in the adventitial or periadventitial region of a vascular structure, a cardiovascular structure, or another organ. The autonomic neural tissue may include, for example, a renal sympathetic nerve, a carotid nerve, a pulmonary nerve, and/or a cardiac sympathetic nerve. The delivery step 202 may be performed using a syringe 203 (FIG. 1), a catheter, or another suitable delivery device. In some embodiments, the delivery step 202 is performed with an injection device comprising a plurality of needles, for example 2, 3, or 4 needles. Delivery step 202 may be performed with a pre-loaded syringe, or may comprise the step of removing the denervation formulation 100 from a vial or other container before delivery. The denervation formulation 100 may also be reconstituted before delivery.

Following the delivery step 202, the sustained-release matrix 104 may gradually degrade and release the denervation drug 102 into the patient in step 204. This gradual release step 204 may occur over an extended time period of several days, weeks, or months, as described further above.

The denervation drug 102 that is released during the gradual release step 204 may ablate the patient's autonomic neural tissue in step 206. This neural ablation step 206 may interrupt or otherwise hinder the transmission of neural signals from the patient's autonomic neural tissue. This neural ablation step 206 may decrease sympathetic nervous system activity and help treat the related cardiovascular disease.

The denervation formulation 100 shown in FIG. 1 and the method 200 shown in FIG. 2 are provided as examples of the various features of the formulation and method and, although the combination of those illustrated features is clearly within the scope of invention, those examples and their illustrations are not meant to suggest the inventive concepts provided herein are limited from fewer features, additional features, or alternative features to one or more of those features shown in FIGS. 1 and 2.

### Test Methods

It should be understood that although certain methods and equipment are described below, other methods or equipment determined suitable by one of ordinary skill in the art may be alternatively utilized.

### Release Profile

For each sample being evaluated, a 700 mL elution media was prepared including 0.5 w/v % sodium dodecyl sulfate, 22 mM sodium acetate, and 28 mM acetic acid (pH 4.6). To a Spectra-Por Float-A-lyzer G2 (Sigma Z727040 MWCO:100 kD) was added 2 mg of the formed microparticles. The Float-A-lyzer was added to a Sotax App 2 dissolution apparatus with 700 mL of media and equilibrated at 37° C. Aliquot samples (1 mL) of media were removed at specific time points (e.g., 4 hours and daily from 1 day to 14 days). The samples were then tested by High Performance Liquid Chromatography (HPLC)-Absorbance spectroscopy using USP protocols.

### EXAMPLES

### Example A: Encapsulation of Paclitaxel into Microparticles Comprising PLA:TMC

### Solutions

PVA/Water Solution: To a 1000 mL media flask was added 25 g of PVA (Mowiol; Sigma 81381) and 1000 g of deionized water.

DCM/Methanol Solution: To a 100 mL vial was added 98 g of DCM (Sigma Aldrich 270997) and 2 g of methanol (Burdick/Jackson LC230-1). The DCM/methanol solution was then shaken vigorously.

PLA:TMC Solution: To a 40 mL vial was added 24.2 g of the DCM/methanol solution followed by 0.650 g of a PLA:TMC copolymer (Gore LT-50).

PLA:TMC/PTX Solution: To another vial in a powder hood was added 0.100 g PTX powder (Indena). The PLA:TMC solution was added to the PTX powder and mixed for 30 minutes.

### Microparticles

Formation: To a 400 mL PTFE beaker (80 mm×106 mm) with a mechanical homogenizer unit (VWR 25D) was added 250 g of the PVA/water solution. The homogenizer unit's probe (VWR 20 mm×125 mm) was inserted into the solution and rotated at a desired rate, specifically 3,015 or 5,035 rpm in this Example. The PLA:TMC/PTX solution was added rapidly to the PVA/water solution to form an emulsion, which was homogenized at the desired rate for 4 minutes. Then, the emulsion was vigorously stirred with a magnetic stir-bar within the isolator exposed to the isolator atmosphere for overnight (12 hours) to allow for formation and hardening of the microparticles and evaporation of residual solvent.

Isolation: To a 50 mL centrifuge tube was added 35 mL of the PLA:TMC/PTX emulsion. The centrifuge was spun at 3,450 rpm for 30 minutes. The centrifuge tube supernate was decanted and the tubes were filled to 35 mL with deionized water. These steps were repeated until the supernate was free of PVA.

Lyophilization: The microparticles were then re-suspended in a minimum amount of deionized water. The microparticle suspension was frozen at −20° C. for a minimum of 4 hours, preferably overnight. The samples were then lyophilized for 24 to 48 hours resulting in dry samples.

### Results

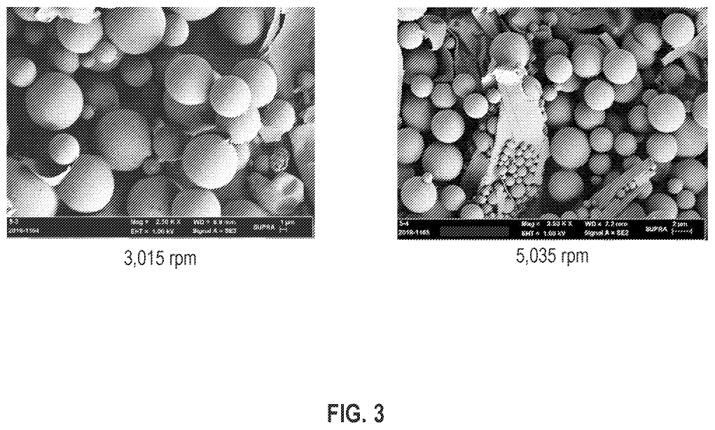

Microparticle Size: The dry PLA:TMC/PTX microparticles were subjected to scanning electron microscope (SEM) imaging, and the results are presented in FIG. 3. The microparticles are spherical in shape and discrete. The microparticles that were formed with slower rotation during emulsification (i.e., 3,015 rpm) were relatively large, with diameters ranging from approximately 1 μm to 5 μm or more. By contrast, the microparticles that were formed with faster rotation during emulsification (i.e., 5,035 rpm) were relatively small, with diameters ranging from approximately 0.5 μm or less to 3 μm.

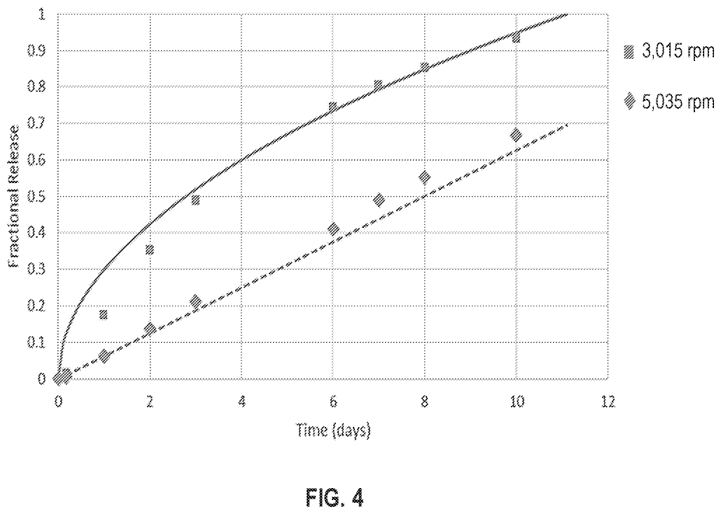

PTX Release Profile: The dry PLA:TMC/PTX microparticles were also subjected to sustained release testing as described above, and the results are presented in FIG. 4. The microparticles that were formed with slower rotation during emulsification (i.e., 3,015 rpm) released PTX at a relatively fast rate, with 100% PTX release after 11 days. This release rate also varied over time, with an initial burst followed by a steady final rate. By contrast, the microparticles that were formed with faster rotation during emulsification (i.e., 5,035 rpm) released PTX at a relatively slow rate, with 60% PTX release after 11 days. It is estimated that This release rate was steady over time.

Based on these results, the speed of rotation during emulsification was shown to have an indirect impact on particle size and an indirect impact on the PTX release rate.

### Example B: Syntheses of Fluorinated Copolymers Comprising Tetrafluoroethylene and Functional Groups Comprising Vinyl Acetate (TFE-VAc)

Copolymers comprising varying mole ratios of vinyl acetate to tetrafluoroethylene (VAc:TFE) were prepared according the following general synthetic scheme. To a nitrogen purged 1 L pressure reactor under vacuum were added 500 g DI water, 2.0 g of 20% aqueous surfactant, 30 ml of distilled vinyl acetate, 10 g of n-butanol, and 0.2 g of ammonium persulfate. Tetrafluoroethylene monomer was then fed into the reactor until the reactor pressure reached 1500 KPa. The mixture was stirred and heated to 50° C. When a pressure drop was observed, 25 ml of additional vinyl acetate was slowly fed into the reactor. The reaction was stopped when the pressure dropped another 150 KPa after vinyl acetate addition. The copolymer was obtained from freeze-thaw coagulation of the latex emulsion, cleaned with methanol/water extraction, and air dried.

The copolymers' composition and molecular weight are listed in Table 1 below.

TABLE 1

| Copolymer# | VAc mole % | TFE mole % | MW(KDa) |
|---|---|---|---|
| 100-0 | 80.0 | 20.0 | 300 |
| 100-1 | 81.1 | 18.9 | 337 |
| 100-2 | 81.2 | 18.8 | 220 |
| 100-3 | 84.5 | 15.5 | 430 |
| 100-4 | 76.5 | 23.5 | 122 |

### Prophetic Example C: Denervation Formulation with Example B

A denervation drug emulsion is added to a solution containing the WE-Vac of Example B by to form an emulsion, through a process similar to Example A, which is homogenized at the desired rate for 4 minutes. Then, the emulsion is vigorously stirred with a magnetic stir-bar within the isolator exposed to the isolator atmosphere for overnight (12 hours) to allow for formation and hardening of the microparticles and evaporation of residual solvent. The emulsion is then lyophilized and isolated as described in Example A.

### Example D: Synthesis of a Fluorinated Copolymer Comprising Tetrafluoroethylene and Functional Groups Comprising Alcohol (TFE-VOH)

The vinyl acetate groups of copolymer #100-0 of Example B were hydrolyzed to vinyl alcohol as follows. To a 50 ml round bottle flask were added 0.5 g of copolymer #100-0 (predissolved in 10 ml methanol) and 0.46 g NaOH (predissolved in 2 mI DI water). The mixture was stirred and heated to 60° C. for 5 hrs. The reaction mixture was then acidified to pH 4, precipitated in DI water, dissolved in methanol, again precipitated in DI water, and air dried. The resulting product was a copolymer of TFE-VOH.

### Example E: Synthesis of Nanoparticles Comprising TFE-VOH

### Nanoparticles

Formation: To a disposable polystyrene cuvette, approximately 900 μL of DI water was added. Nanoparticles were immediately formed by adding approximately 100 μL TFE-VOH from Example D to DI water.

### Results

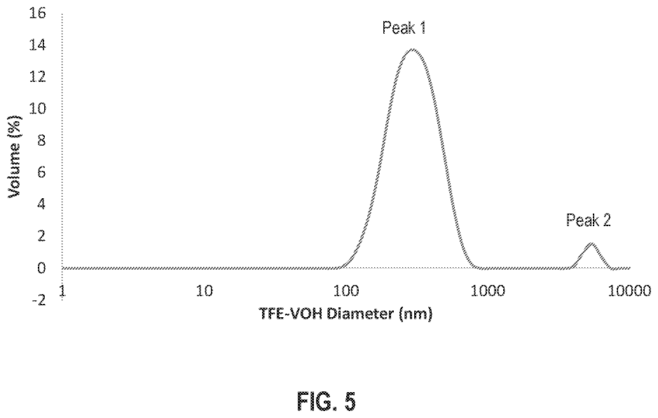

Nanoparticle Size: The cuvette containing the TFE-VOH nanoparticles was added to a Malvern ZetaSizer Ultra and diameter (or Z-Average) was obtained via dynamic light scattering (DLS). FIG. 5 presents the representative volume distribution of the solution indicating that a large fraction of the composition contains nanoparticles (Peak 1) and a small fraction of the composition (<6.0% average) (Peak 2) contains unformed nanoparticles, which may be removed via centrifugation. The physical properties of the composition are summarized in Table 2, where volume % describes the relative proportion of particles in the composition. The average nanoparticle diameter was 295±7.2 nm, and the average polydispersity index (PDI) was 0.29±0.06.

TABLE 2

| Property | Average | Std. Dev. | Min | Max |
|---|---|---|---|---|
| Diameter (nm) | 295 | 7.2 | 290 | 303 |
| Peak 1 Volume (%) | 97.9 | 2.5 | 95.1 | 100 |
| Peak 2 Volume (%) | 3.17 | 2.49 | 0 | 4.93 |

Nanoparticle Chemical Properties: The TFE-VOH solution from Example D (Spectra A) and nanoparticles from this Example E (Spectra B) were added to a Nicolet 6700 FTIR with ATR for chemical characterization. FIG. 7 shows equivalent Spectra A and B, which suggests structural integrity after nanoparticle formation.

### Prophetic Example F: Denervation Formulation with Example E

A denervation drug emulsion is added to a solution containing the TVE-VOH of Example E by to form an emulsion, through a process similar to Example A, which is homogenized at the desired rate for 4 minutes. Then, the emulsion is vigorously stirred with a magnetic stir-bar within the isolator exposed to the isolator atmosphere for overnight (12 hours) to allow for formation and hardening of the microparticles and evaporation of residual solvent. The emulsion is then lyophilized and isolated as described in Example A.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A formulation comprising:
a sustained-release matrix comprising at least one of a polycarbonate and a fluoropolymer; and
a denervation drug incorporated into the sustained-release matrix;
wherein the sustained-release matrix is configured to release the denervation drug at a release rate of 1 μg/day to 700 μg/day.

2. The formulation of claim 1, wherein the denervation drug comprises at least one of paclitaxel, vincristine sulfate, vinblastine, digoxin, and analogs and salts thereof.

3. The formulation of claim 1, wherein the sustained-release matrix forms a plurality of particles that encapsulate the denervation drug.

4. The formulation of claim 3, further comprising a delivery fluid mixed with the plurality of particles to form an injectable formulation.

5. The formulation of claim 3, wherein each particle has a diameter of 0.1 μm to 20 μm.

6. (canceled)

7. The formulation of claim 1, wherein the sustained-release matrix is configured to release the denervation drug over a time period of 3 days to 180 days.

8.-10. (canceled)

11. The formulation of claim 1, further comprising at least one excipient comprising at least one of sodium glutamate, decylmethyl sulfoxide and calcium salicylate.

12. The formulation of claim 1, wherein the sustained-release matrix comprises at least one of:
(a) a trim ethylene carbonate (TMC) moiety and at least one of a polylactic acid (PLA) moiety and a polyglycolic acid (PGA) moiety; and
(b) a tetrafluoroethylene (TFE) moiety and a vinyl moiety, wherein the vinyl moiety comprises at least one functional group selected from acetate, alcohol, amine, and amide.

13. (canceled)

14. The formulation of claim 1, wherein the sustained-release matrix is at least one of a solid and a gel.

15. A denervation method comprising:
delivering a denervation formulation to an autonomic neural tissue of a patient with a disease, the denervation formulation comprising a denervation drug incorporated into a sustained-release matrix forming a plurality of particles that encapsulate the denervation drug;
gradually releasing the denervation drug into the autonomic neural tissue of the patient; and
ablating the autonomic neural tissue.

16. The method of claim 15, wherein the autonomic neural tissue is at least one of a renal sympathetic nerve, a carotid nerve, a pulmonary nerve, and a cardiac sympathetic nerve;
wherein the autonomic neural tissue is located in at least one of an adventitial region of a vascular structure, a periadventitial region of a vascular structure, and a cardiovascular structure;
wherein delivering the denervation formula is performed using one of a syringe and a catheter; and
wherein the disease is at least one of hypertension, heart failure, type II diabetes, pulmonary arterial hypertension, fatty liver disease, sleep apnea, chronic kidney disease atrial tachycardia, and ventricular tachycardia.

17.-19. (canceled)

20. The method of claim 15, wherein the releasing step occurs at a rate of 0.1 μg/day to 2 μg/day over a time period of 5 days to 15 days.

21. (canceled)

22. The method of claim 15, wherein the sustained-release matrix comprises at least one of:
(a) a poly(lactic acid-trimethylene carbonate) copolymer (PLA:TMC); and
(b) a tetrafluoroethylene (TFE) moiety and a vinyl moiety, wherein the vinyl moiety comprises at least one functional group selected from acetate, alcohol, amine, and amide.

23. (canceled)

24. The formulation of claim 1, wherein the sustained-release matrix comprises an amphiphilic block copolymer.

25. (canceled)

26. The formulation of claim 1, wherein the sustained-release matrix comprises at least one of a polylactic acid (PLA) moiety, a polyglycolic acid (PGA) moiety, a polyethylene glycol moiety, and a trim ethylene carbonate (TMC) moiety.
